(19) United States
(12) Patent Application Publication
(10) Pub. No.: US 2023/0317224 A1
(43) Pub. Date: Oct. 5, 2023

(54) PATIENT SPECIFIED HEALTH RECORD ON BLOCKCHAIN

(71) Applicant: MatrixCare, Inc., Bloomington, MN (US)

(72) Inventor: Adhiraj Ganpat PRAJAPATI, St. Paul, MN (US)

(21) Appl. No.: 17/707,711

(22) Filed: Mar. 29, 2022

(51) Int. Cl. G16H 10/65; G06Q 20/36; H04L 9/30

(52) U.S. Cl. CPC G16H 10/65; H04L 2209/38; H04L 9/30; G06Q 20/363

(57) ABSTRACT

Embodiments herein use a blockchain to store encrypted patient data. The blockchain may be distributed amongst multiple different healthcare providers and use a shared data format standard. By placing the patient data on a blockchain which is accessible by multiple healthcare providers, the data is more readily transferrable. The patient can maintain a EHR wallet that stores cryptography elements for decrypting that patient's data on the blockchain. For example, when the patient wants her data to be readable by a healthcare provider that did not put the data on the blockchain, the patient can transfer the corresponding cryptography element (e.g., a private key) from her wallet to the new healthcare provider who can then use the cryptography element to decrypt the patient's data that was put on the blockchain by a different healthcare provider.

### BACKGROUND

### Field

Embodiments of the present invention generally relate to storing patient healthcare data on a blockchain.

### Description of the Related Art

Currently, many different software vendors are used to generate healthcare records (e.g., electronic health records (EHR)). These vendors typically use different formats and techniques for generating the EHRs, which makes transferring EHRs between two healthcare providers that use different software vendors for managing EHRs a difficult task. The vendors either have to offer a software tool to convert the EHR generated by one vender into a format accepted by the other vendor, or the information in the EHR has to be manually entered into the application used by the new provider. Because most software vendors do not have the tools needed to convert the format, transferring patient data between healthcare providers is often a labor-intensive task and is susceptible to human errors. This often results in mistakes and critical omissions in transferred EHRs.

### SUMMARY

One embodiment herein is a method that includes transmitting, from a first healthcare provider, a request to access encrypted patient data put on a blockchain by a second healthcare provider, receiving a cryptography element from an EHR wallet controlled by a recipient of the request, identifying the encrypted patient data on the blockchain, and decrypting the encrypted patient data using the cryptography element.

Another embodiment herein is a method that includes adding, by a first healthcare provider, first encrypted patient data for a first patient to a first block in a blockchain; adding, by the first healthcare provider, second encrypted patient data for a second patient to a second block in the blockchain; and distributing the blockchain, which contains the first and second blocks, to a plurality of healthcare providers.

Another embodiment herein is a non-transitory computer readable medium comprising instructions to be executed in a processor, the instructions when executed in the processor perform an operation. The operation comprising transmitting, from a first healthcare provider, a request to access encrypted patient data put on a blockchain by a second healthcare provider; receiving a cryptography element from an EHR wallet controlled by a recipient of the request; identifying the encrypted patient data on the blockchain; and decrypting the encrypted patient data using the cryptography element.

Another embodiment herein is a method that includes adding, by a first healthcare provider, first encrypted patient data for a first patient to a first block in a blockchain; providing a key to a second healthcare provider or a group of healthcare providers to grant access to the first encrypted patient data; and distributing the blockchain, which contains the first block, to a plurality of healthcare providers.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 illustrates healthcare providers adding patient data to a blockchain, according to one embodiment.

FIG. 2 illustrates computing systems for managing a blockchain, according to one embodiment.

FIG. 3 illustrates a blockchain, according to one embodiment.

FIGS. 4A and 4B are flowcharts for adding encrypted patient data to a blockchain, according to one embodiment.

FIG. 5 is a flowchart for enabling a healthcare provider to access encrypted patient data on the blockchain, according to one embodiment.

FIG. 6 is a flowchart for adding different types of encrypted patient data to a blockchain, according to one embodiment.

FIG. 7 illustrates a blockchain and an EHR wallet with keys for accessing patient data on the blockchain, according to one embodiment.

FIG. 8 is a flowchart for enabling a healthcare provider to access encrypted patient data on the blockchain, according to one embodiment.

FIG. 9 illustrates decrypting different types of patient data on the blockchain, according to one embodiment.

FIG. 10 illustrates decrypting different types of patient data on the blockchain, according to one embodiment.

FIG. 11 is a flowchart for enabling an organization to access encrypted patient data on the blockchain, according to one embodiment.

FIG. 12 illustrates a computing system, according to one embodiment.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments herein use a blockchain to store encrypted patient data (e.g., data that is part of an EHR). The blockchain may be distributed to multiple different healthcare providers and use a shared data format standard such as the Fast Healthcare Interoperability Resources (FHIR) standard. By placing the patient data on a blockchain that is accessible by multiple healthcare providers, the data is more readily transferrable. Each patient can maintain an EHR wallet (like a virtual currency wallet) that stores cryptography elements for decrypting that patient's data on the blockchain. For example, when the patient wants her data to be accessible to a healthcare provider that did not put the data on the blockchain, the patient can transfer the corresponding cryptography element (e.g., a private key) from her wallet to the new healthcare provider who can then use the cryptography element to decrypt the patient's data that was put on the blockchain by a different healthcare provider. In this manner, the patient data is encrypted and is immutably stored on the blockchain, but can also be made accessible (or transferred) to healthcare providers who did not put the data on the blockchain.

In additional embodiments, the patient can control what types of patient data is made accessible or transferred to the healthcare provider. For example, the patient may want the new healthcare provider to see only diagnosis data (e.g., the results of a medical test) but not her treatment data (e.g., prescribed medications or surgeries she underwent). The different types of patient data can be encrypted on the blockchain using different cryptography elements (e.g., different keys). The patient can provide the healthcare provider with a key that can be used to decrypt only a certain type of data (e.g., the diagnosis data but not the treatment data). Thus, after receiving the key, the healthcare provider can decrypt only the diagnosis data stored on the blockchain, but not any of the other types of the patient's data on the blockchain. In this manner, the patient has control over when and what type of data is transferred between healthcare providers.

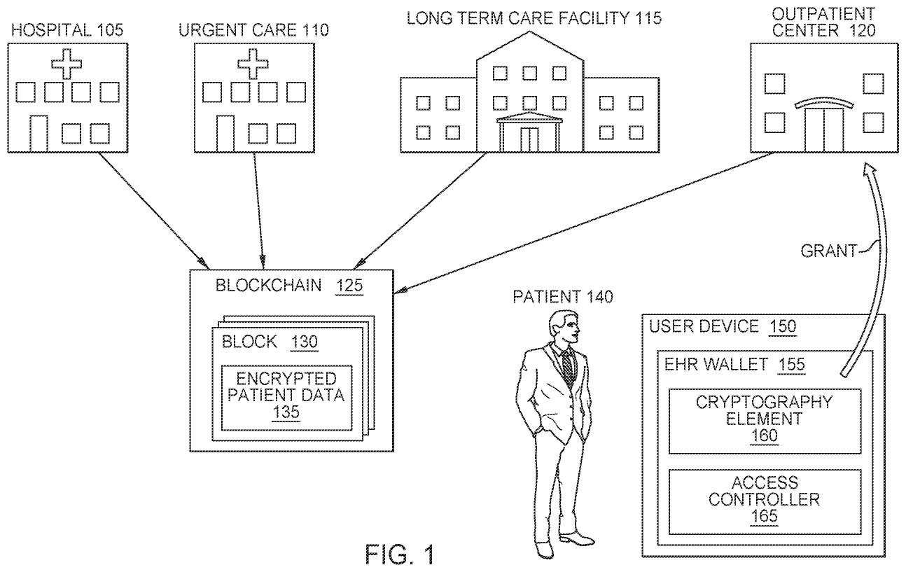

FIG. 1 illustrates healthcare providers adding patient data to a blockchain, according to one embodiment. FIG. 1 illustrates several examples of a healthcare provider such as a hospital 105, urgent care 110, long term care facility 115, and outpatient center 120. However, these are just some of the examples of a healthcare provider who might put patient data on a blockchain 125. Further, a healthcare provider can include multiple facilities that are part of the same healthcare system, whether a private healthcare system or a government healthcare system.

The healthcare providers can add encrypted patient data 135 to the blockchain 125. The patient data 135 can be data that is typically added to an EHR for the patient. For example, the healthcare providers may add diagnostic information, prescriptions, diagnoses, surgeries, treatment plans, and the like to the blockchain 125. In one embodiment, the size of the blocks 130 in the blockchain 125 may be a set size. Once the healthcare provider collects enough patient data 135 (which can be for the same patient or for multiple patients), the provider adds a new block 130 to the blockchain 125. The healthcare providers can add new blocks 130 to the blockchain 125 in parallel. Further, these blocks 130 can be shared and stored at computer systems corresponding to the different healthcare providers shown in FIG. 1.

In one embodiment, the blockchain 125 is a distributed database that is shared among the healthcare providers. That is, the healthcare providers can serve as nodes of a computer network that store the blocks 130 and patient data 135 that make up the blockchain 125. The blockchain 125 maintains a secure and decentralized record of transactions where patient data 135 is added to the blockchain. The blockchain 125 can guarantee the fidelity and security of the encrypted patient data 135 without the need for a trusted third party.

The healthcare providers can collect the patient data 135 in groups—i.e., the blocks 130—that have certain storage capacities that, when filled, are closed and linked to the previous block 130 in the blockchain 125. The chain of blocks 130 forms a timeline of data where each block 130 can be given a timestamp indicating when it was added to the blockchain 125. In one embodiment, the healthcare providers can add and distribute the data in the blockchain but cannot edit it. For example, the blockchain 125 can form a distributed ledger where the patient data cannot be altered, deleted, or removed from the chain without permission from all (or a majority) of the healthcare providers.

As shown, each of the healthcare providers can add blocks 130 that contain patient data 135 to form EHRs for their patients. However, because the data is encrypted, this means a healthcare provider (or a nefarious actor who obtains a copy of the blockchain 125) cannot decrypt the patient data that was added by another provider. That is, although the blockchain 125 is distributed to all the healthcare providers, the hospital 105 cannot decrypt the patient data added to the blockchain by the urgent care 110, the long term care facility 115, or the outpatient center 120, and vice versa. Nonetheless, even though the data is encrypted, the blocks 130 can still include hashes that the healthcare providers can use to determine if one of the distributed copies of the blockchain 125 has been tampered with, and thus, should be discarded. Thus, ensuring the blockchain 125 is not altered by one healthcare provider does not require all the providers to be able to decrypt and read the patient data 135. In this manner, the healthcare providers can ensure the blockchain 125 is immutable while maintaining patient privacy by keeping the patient data in an encrypted state.

A patient 140 can have a user device 150 (e.g., a smart phone, laptop, tablet, etc.) that includes an EHR wallet 155 that stores one or more cryptographic elements 160 (e.g., a private key or keys) for decrypting the encrypted patient data 135 on the blockchain 125. In one embodiment, when one of the healthcare providers adds encrypted data 135 to the blockchain 125 for the patient 140, the provider also sends a cryptographic element 160 to the EHR wallet 155 for decrypting that data 135. Doing so provides the patient 140 with the means to decrypt her own data of the blockchain 125. Further, the patient 140 can send the cryptographic element 160 to a different healthcare provider so that provider also has the means to decrypt and read the patient's data on the blockchain 125.

Although the embodiments herein describe the EHR wallet 155 being part of a device controlled by the patient, in other embodiments the EHR wallet 155 may be stored or controlled by one of the healthcare providers. In that case, the healthcare provider that controls the EHR wallet 155 for the patient may still wait for patient permission before it transmits a cryptographic element stored in the wallet 155 to another healthcare provider.

In this example, the user device 150 include an access controller 165 which can be a software application that permits the patient 140 to send the cryptographic element 160 to a healthcare provider. The access controller 165 can receive requests from one of the healthcare providers to access the patient's data on the blockchain. For example, the patient 140 may be seeking a second opinion from a doctor at a different healthcare provider, or the patient 140 may be in the process of being transferred from the hospital 105 to the long term care facility 115. In response, the new healthcare provider can submit a request to the access controller 165 for access to the patient's encrypted patient data 135 on the blockchain 125.

The access controller 165 can use a display screen and input/output (IO) elements in the user device 150 (e.g., a touch screen, mouse, or keyboard) to inform the patient 140 of the request and ask for the patient's permission. The access controller 165 can use enhanced security protocols to make sure the patient has given permission such as collecting biometric data, asking for a password or pin, or performing two-factor authorization. Doing so can ensure that the user device 150 receives permission from the patient 140 rather than a nefarious actor who has obtained control of the user device 150.

Once the patient 140 gives permission, the access controller 165 can send the cryptography element 160 (e.g., a private key) to the healthcare provider. FIG. 1 illustrates the access controller 165 sending the cryptography element 160 to the outpatient center 120. The outpatient center 120 can then decrypt the encrypted patient data 135 for the patient 140 on the blockchain 125.

In one embodiment, the cryptography element 160 can be used to decrypt all the patient's data on the blockchain 125, regardless of which healthcare provider added the patient's data to the blockchain 125. For example, if the outpatient center 120 adds another block 130 to the blockchain 125 which has additional data for the patient 140, this data may be encrypted using the same cryptography element (e.g., the same public key) as data that was previously added to the blockchain 125 by a different healthcare provider. That way, the same cryptography element 160 (e.g., the same private key) in the EHR wallet 155 can be used to decrypt the patient's data, regardless whether that data was added from the same healthcare provider or from different providers.

However, in another embodiment, the healthcare providers may use different keys when encrypting patient data and adding that data to the blockchain 125. In that case, the EHR wallet 155 may store separate cryptography elements 160 (e.g., different private keys) for decrypting her data. If the patient 140 sends both cryptography elements 160 to a new healthcare provider, this enables the new provider to decrypt and view the patient's data added to the blockchain by both of her old healthcare providers. However, the patient 140 may send only one cryptography element 160 to the new healthcare provider which enables the new provider to decrypt and view the data put on the blockchain 125 from only one of the old healthcare providers. Having separate cryptography elements 160 for the data 135 put on the blockchain 125 from different healthcare providers gives the patient 140 more granular control of her data. The patient 140 can then choose to share all her data with a new healthcare provider, or only a subset of the data (e.g., the data put on the blockchain 125 for a subset of all the healthcare providers that put medical information of the patient 140 on the blockchain 125). Moreover, as discussed in later figures, the patient data can be further subdivided into different types of data, and the patient can control which types of medical data is transferred to a new healthcare provider.

In general, FIG. 1 illustrates a blockchain system where multiple different healthcare providers can add patient data for multiple patients to a shared, distributed blockchain 125. The patient data for each patient can be encrypted. Further, each patient can maintain an EHR wallet 155 with one or more cryptography elements 160 that can be shared with other healthcare providers to essentially “transfer” some or all of the encrypted data 135 for the patient 140 to the new healthcare provider.

The EHR wallet 155 can be implemented using a variety of different hardware and software strategies. In one embodiment, the EHR wallet 155 can be a desktop wallet that is installed on a desktop or laptop computer that provides the patient 140 with complete control over the wallet 155. In another embodiment, the EHR wallet 155 can be a mobile wallet that is installed on a smartphone or other mobile device. The mobile wallet can facilitate quick transfers of the cryptography element 160 when at a new healthcare provider through near field communication (NFC) or by scanning a QR code containing the element 160. In another embodiment, the EHR wallet 155 can be a web wallet such as an online service that can send and store the cryptography elements 160 on behalf of the patient 140. The main advantage of web wallets is that they can be accessed anywhere, from any device. In another embodiment, the EHR wallet 155 can be a hardware wallet which stores the cryptography elements 160 on a physical device that cannot access the Internet. The physical device (which can resemble a USB drive) can be plugged into another computer device when the patient 140 wishes to grant a new healthcare provider with access to some or all of her EHR stored on the blockchain 125.

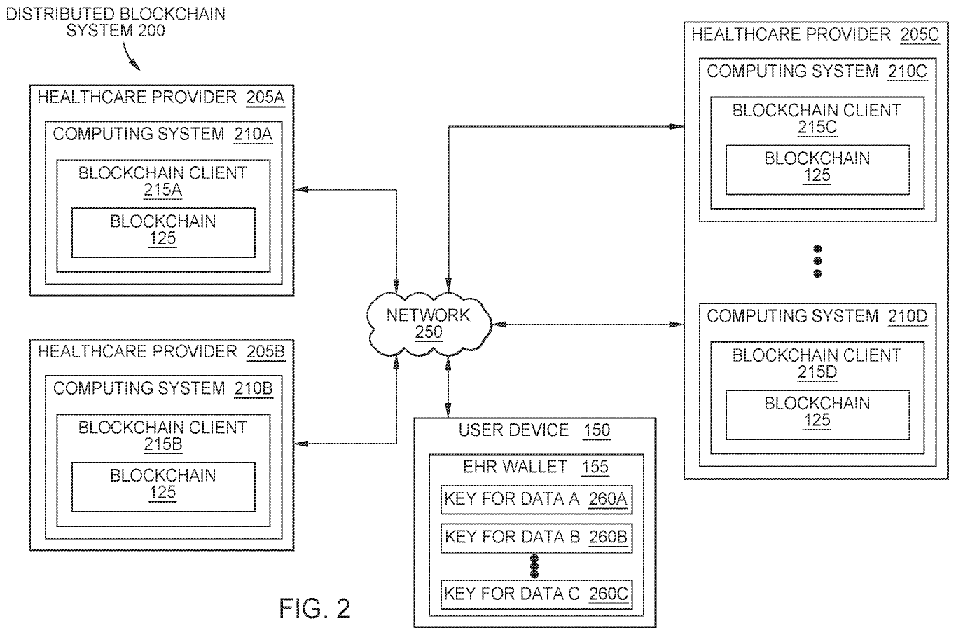

FIG. 2 illustrates computing systems 210 for managing a distributed blockchain system 200, according to one embodiment. In the illustrated embodiment, the system 200 includes a user device 150 and three healthcare providers 205A-C. Although three healthcare providers 205A-C are illustrated, any number of providers may be included in the network. Similarly, although a single user device 150 is depicted, the system can include as many user devices as there are patients for the healthcare providers 205A-C.

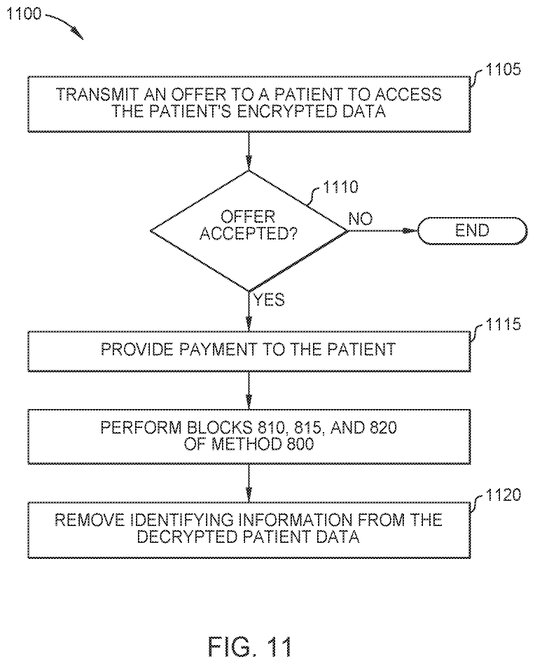

In the illustrated embodiment, the healthcare providers 205 include at least one computing system 210 which can include a processor and memory (which are discussed in FIG. 11). The healthcare providers 205A and 205B include one computing system 210, while the healthcare provider 205C includes multiple computing systems 210. For example, the healthcare providers 205A and 205B may be healthcare facilities such as a hospital or outpatient facility while the healthcare providers 205 can be a healthcare system with multiple healthcare facilities that each has a computing system 210. In general, the computing systems 210 are tasked with collecting patient data, adding encrypted patient data to the blockchain 125, and maintaining distributed copies of the blockchain 125.

The computing systems 210 each include a blockchain client 215 that maintains a copy of the blockchain 125. Further, the blockchain client 215 (e.g., a software application) can add blocks of patient data to the blockchain 125. In addition, the blockchain client 215 can distribute newly added blocks to the other healthcare providers 205 so their copies of the blockchain 125 have the newly added data. That is, in an embodiment, when a healthcare provider 205 generates updated patient data that should be added to the patient's EHR, the blockchain client 215 generates a record of the updated data, updates its ledger to reflect the change, and broadcasts the change to the computing systems 210 of the other healthcare providers 205.

In an embodiment, each respective blockchain client 215 attempts to solve a defined algorithm, or otherwise verify a predefined criterion, that triggers the end of the current block. Upon determining that the criteria is satisfied, the respective blockchain client 215 transmits an indication to the other computing systems 210, which verify the solution and accept the new block. In this way, each time a new block of patient data is generated by one healthcare provider 205, it is recorded by each healthcare provider 205.

The network 250 also permits the user device 150 for a patient to receive keys that can be used to decrypt that patient's data on the blockchain 125. In this embodiment, the EHR wallet 155 stores a different key for data added to the blockchain by the different healthcare providers 205. While it is possible for one key to be used to decrypt all the patient's data (regardless which healthcare provider 205 put the data on the blockchain 125), in this embodiment, the EHR wallet 155 has a different key for different chunks of the patient's data. For example, a key 260A for Data A can be used to decrypt the patient's data put on the blockchain by healthcare provider 205A, while the key 260A for Data B can be used to decrypt the patient's data put on the blockchain by healthcare provider 205B, and the key 260C for Data C can be used to decrypt the patient's data put on the blockchain by healthcare provider 205C.

As discussed above, the patient can send the keys 260 to a different healthcare provider than the one that put the data on the blockchain 125 so that provider can access or read the data. For example, the patient can send the key 260A for Data A (which was put on the blockchain 125 by the provider 205A) to the healthcare provider 205B or 205C so it can read or access this data. Without the key 260A, these healthcare providers 205B and 205C are unable to decrypt Data A. A similar strategy can apply to the keys 260B and 260C where those keys can be sent (after obtaining the patient's authorization) to healthcare providers who did not put Data B and Data C on the blockchain so those providers can access this data.

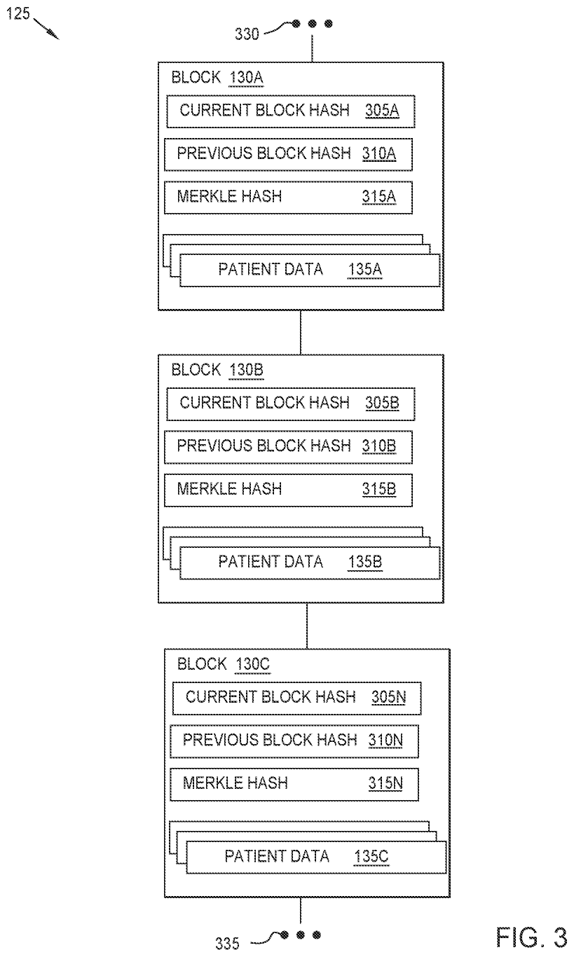

FIG. 3 illustrates one example of the blockchain 125, according to one embodiment. As shown, FIG. 3 illustrates several blocks 300A-N in the blockchain 125. Although three blocks are illustrated, as indicated by the ellipses 330 and 335, the blockchain 125 may include any number of blocks preceding and following the illustrated blocks 130A-C. As illustrated, in an embodiment, each block 130 in the blockchain 125 includes a respective current block hash 305A-N, a respective previous block hash 310A-N, and a respective Merkle hash 315A-N. Further, as illustrated, each respective block 130 includes a respective set of patient data 135A-C.

In an embodiment, the current block hash 305A includes a hash value representing the block 130A. In some embodiments, the current block hash 305A may be based in part on the previous block hash 310A and the Merkle hash 315A. In some embodiments, the current block hash 305A is also based in part on a nonce value, or other data. Further, in the illustrated embodiment, the previous block hash 310A refers to the hash of the previous block in the chain. For example, the previous block hash 310B in the block 130B has the same value as the current block hash 305A in the block 130A. Similarly, the previous block hash 310C in the block 130C has the same value as the current block hash 305B in the block 130B.

In the illustrated embodiment, the Merkle hash 315 of the blocks 130 is based on each of the patient data 135A-C included in the respective block 130A-C. For example, in one embodiment, each of the patient data 135A-C included in the ledger of the blocks 130A-C is hashed, and the resulting values are iteratively combined and hashed until a final value (e.g., a Merkle hash 315A-C) representing all of the patient data 135A-C in the blocks 130A-C is created. In this way, none of the individual patient data 135A-C can be modified or removed, as this would change the Merkle hash 315A. Further, in some embodiments, a change in the Merkle hash 315A would cause the current block hash 305A to change as well. This, in turn, would cause the hash of each subsequent block (e.g., current block hash 305B) to change. In this way, the blockchain 125 is secure, consistent, and immutable.

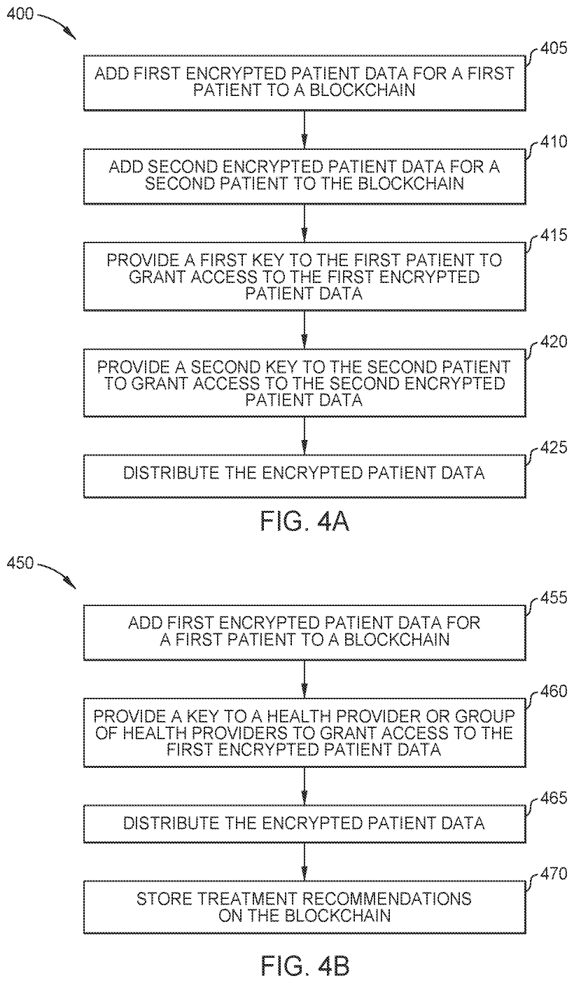

FIG. 4A is a flowchart of a method 400 for adding encrypted patient data to a blockchain, according to one embodiment. At block 405, a first healthcare provider adds first encrypted patient data for a first patient to a blockchain. The first healthcare provider can add the first encrypted patient data into a block, which is then added to the blockchain. The block can include only medical data for the first patient or the block can also include encrypted medical data for other patients being treated at the first healthcare provider. Stated differently, each block added to the blockchain can include patient data for one patient, or for multiple patients. In one embodiment, the first healthcare provider adds a new block each time it collects a certain size (e.g., 100 megabytes) of patient data.

If the first encrypted data is put in a block in the blockchain with encrypted data for other patients, the data may be decrypted using different cryptography elements. For example, the first healthcare provider may use a different key to encrypt data for different patients. In one embodiment, the first healthcare provider may have a different public key for each patient which it uses to encrypt any medical data for that patient. The EHR wallet for each patient can have a different private key for decrypting their data. Thus, even if the block has encrypted data for multiple patients, a private key from one of the patients will enable a new healthcare provide to decrypt and read only that patient's medical data in the block, but not the encrypted data for other patients also stored in the same block.

While the method 400 discusses using private and public key pairs to encrypt and decrypt patient data on the blockchain, this is just one example. The embodiments herein can be used with any cryptographic technique where a patient can store in a wallet a cryptography element that can be shared with other healthcare providers so they can access the patient's medical records in the blockchain.

At block 410, the first healthcare provider adds second encrypted patient data for a second patient to the blockchain. The second encrypted patient data can be stored in the same block as the first encrypted patient data, or the first healthcare provider may add the second encrypted patient data to a different block which is connected to the block containing the first encrypted patient data (like the blockchain 125 illustrated in FIG. 3). In any case, in one embodiment, the first and second encrypted patient data cannot be decrypted using the same cryptographic element (e.g., the same key). Instead, the healthcare provider may ensure the first and second encrypted patient data can only be decrypted using different keys (e.g., different private keys assigned to the first and second patients). That way, even though the medical records for multiple patients are stored in the same blockchain, the medical records for a particular patient are accessible only to that patient, or to someone who is sent the key for decrypting the medical records by the patient.

At block 415, the first healthcare provider provides a first key to the first patient to grant access to the first encrypted patient data. In one embodiment, when first collecting medical data for the first patient to put on the blockchain, the first healthcare provider may generate a public-private key pair for the patient. The first healthcare provider can then use the public key to encrypt the first patient's data each time it is added to the blockchain. The first healthcare provider can then provide the private key to the first patient, which the first patient can store in their EHR wallet.

Alternatively, the patient may generate the public-private key pair. For example, the access controller on the patient's user device (e.g., the user device 150 in FIG. 1) may generate the key pair. In that case, the access controller can send the public key to the first healthcare provider to use when encrypting the first encrypted patient data at block 405.

In either case, the patient's EHR wallet contains the key for decrypting the first encrypted patient data. Moreover, this key may be unable to decrypt other patient's data stored in the blockchain, such as the second encrypted data for the second patient.

At block 420, the first healthcare provider provides a second key to the second patient to grant access to the second encrypted patient data. In one embodiment, when first collecting medical data for the second patient to put on the blockchain, the first healthcare provider may generate a public-private key pair for the patient. The first healthcare provider can then use the public key to encrypt the second patient's data each time it is added to the blockchain. The first healthcare provider can then provide the private key to the second patient, which the second patient can store in their EHR wallet.

Alternatively, the second patient may generate the public-private key pair. For example, the access controller on the second patient's user device (e.g., the user device 150 in FIG. 1) may generate the key pair. In that case, the access controller can send the public key to the first healthcare provider to use when generating the second encrypted patient data at block 410. In any case, the EHR wallet for the second patient has the second key so the patient (or anyone the patient sends the key to) can access the second encrypted data on the blockchain.

At block 425, the first healthcare provider distributes the first and second encrypted patient data to the other healthcare providers in the network. That way, these healthcare providers can add the first and second patient data to their copies of the distributed blockchain. As discussed above, this helps to ensure the blockchain is immutable from nefarious actors who may wish to alter or delete some of the patient data. However, it is still possible for the healthcare providers to delete or alter the patient data. For example, to comply with privacy laws (or at the behest of a patient), the healthcare providers can vote to delete old patient data from the blockchain. This may cause the healthcare providers to then update the hashes discussed in FIG. 3. While the healthcare providers can alter the blockchain after a vote, a nefarious actor cannot.

FIG. 4B is a flowchart of a method 450 for adding encrypted patient data to a blockchain, according to one embodiment. Rather than distributing the encryption keys to a patient, in the method 450 the keys are distributed directly to one or more healthcare providers (e.g., physicians) that have an established relationship with the patient. The method 450 can be used independently of, or in parallel with, the method 400 in FIG. 4A.

At block 455, a first healthcare provider adds first encrypted patient data for a first patient to a blockchain. The first healthcare provider can add the first encrypted patient data into a block, which is then added to the blockchain. The block can include only medical data for the first patient or the block can also include encrypted medical data for other patients being treated at the first healthcare provider. Stated differently, each block added to the blockchain can include patient data for one patient, or for multiple patients. In one embodiment, the first healthcare provider adds a new block each time it collects a certain size (e.g., 100 megabytes) of patient data.

In this embodiment, the first patient data is intended to be accessed by a specific physician or a group of physicians. For example, the physician may be the primary care physician of the patient, or a physician who the patient is using to obtain a second opinion. The first encrypted data can include all of the medical data of the first patient, or only a subportion of the patient's medical data. For example, if the physician is the patient's primary care physician, then the method 450 can be used to provide the physician with access to all of the patient's data. However, if the designated physician is a specialist, the method 450 can be used to provide the specialist access only to relevant patient medical data. For example, a healthcare provider can use the method 450 to provide a hematologist with the portion of a patient's medical data associated with blood, blood-forming organs and blood diseases but the encrypted in block 455 does not include medical data associated with, for example, the patient's muscular system. For instance, the medical provider can use the method 450 to provide access to the relevant medical data (e.g., blood diseases) for each of the hematologist's patients.

In another example, the encrypted patient data is intended to be shared with a group of physicians that are tasked to come to a consensus on how to treat the patient. For example, a patient may have a complex ailment where multiple physicians (e.g., a panel of physicians) recommend a treatment. These physicians can be dispersed at multiple different geographic locations. The patient can use the method 450 to provide the physicians with access to her medical data (or a sub portion of the data) on the blockchain. In one embodiment, each of the physicians on the panel can access all the medical records of the patient. However, in other embodiment, the method 450 can provide customized medical data to each of the physicians on the panel so that they receive access only to the medical data that relates to their specialty.

At block 460, the first healthcare provider provides a first key to the healthcare provider (e.g., a specific physician), a first key to a group of healthcare providers (e.g., a panel of physicians) to grant access to the first encrypted patient data, or a discrete key unique to each of the healthcare providers. In one or more embodiments, the first healthcare provider can provide a private key to the physician or group of physicians, which they can store in their EHR wallets. While the method 450 discusses using private and public key pairs to encrypt and decrypt patient data on the blockchain, this is just one example.

At block 465, the first healthcare provider distributes the first encrypted patient data to the other healthcare providers in the network. That way, these healthcare providers can add the first patient data to their copies of the distributed blockchain. As discussed above, this helps to ensure the blockchain is immutable from nefarious actors who may wish to alter or delete some of the patient data.

At block 470, the healthcare provider or the group of healthcare providers that received the key or keys at block 460 store treatment recommendations on the blockchain, where, in one or more embodiments, this can be used by the healthcare provider to then indicate the treatment being recommended for the patient. That is, the healthcare provider can use the key to retrieve the encrypted data, review it, and provide a recommendation, including treatments and/or prophylactic measures intended to prevent the patient from sustaining harm. In one or more embodiments, if the key or keys are provided to a group of physicians, they can save their recommendations (e.g., votes) on the blockchain. Once the physicians achieve a consensus, this prophylactic measure and/or treatment can then be used for the benefit of the patient. In one example, the treatment may first be sent to the patient who approves the treatment. Alternatively, the treatment may be sent to a primary physician who approves the treatment.

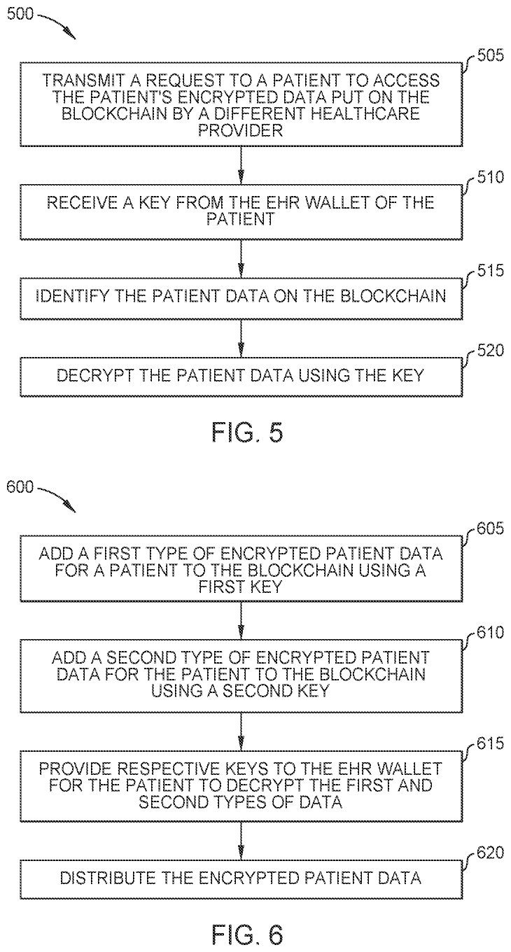

FIG. 5 is a flowchart of a method 500 for enabling a healthcare provider to access encrypted patient data on the blockchain, according to one embodiment. At block 505, a healthcare provider transmits a request to a patient to access the patient's encrypted data put on the blockchain by a different healthcare provider. Because the patient data was put on the blockchain by a different healthcare provider, the requesting healthcare provider would not have access to the encrypted data. For example, the patient may be seeking a second opinion from a doctor at a different healthcare provider, or the patient may be in the process of being transferred from a hospital to a long term care facility. In response, the new healthcare provider can submit a request at block 505 to the patient for access to the patient's encrypted patient data.

At block 510, the healthcare provider receives a key from the EHR wallet of the patient. That is, because the patient can maintain the EHR wallet with the necessary keys (or more generally, cryptography elements), the patient can approve (or not approve) a request to access her encrypted data on the blockchain from a different healthcare provider. Thus, the embodiments herein give the patient direct control, via the EHR wallet, of her encrypted medical data on the blockchain. In one embodiment, the patient may be the only actor with the ability to permit another entity to decrypt her medical data from the blockchain. For example, the original healthcare provider may be unable to provide the key to the new healthcare provider for decrypting the patient's data on the blockchain.

To approve the request, the patient can use an access controller (e.g., the access controller 165 on the user device 150 in FIG. 1) to send the key to the requesting healthcare provider. This key can be itself be encrypted or sent using a secure transaction to the healthcare provider.

In one embodiment, the key includes an attribute that permits the healthcare provider (e.g., the physician) to share the patient data with another physician. That is, the attribute can permit the physician to forward the key to another physician who can then access the patient data. For example, the physician may want a second opinion from a colleague, or may want to forward the data to a panel of physicians who can then vote on treatment recommendations, as discussed in the method 450 in FIG. 4B.

However, if the attribute is not present, then the physician cannot forward the key to another healthcare provider. Nonetheless, if the physician wants permission to share the patient's medical data, the physician could send another request to the patient (at block 505) but this time request that the key has the attribute so the physician can forward the key. For example, when sending the updated request, the physician can tell the patient the reason for wanting to share her patient data with another provider. The patient can then approve or deny the request.

At block 515, the healthcare provider identifies that patient data on the blockchain. For example, the healthcare provider can use a search feature or labels to identify the patient's medical data on the blockchain. Because the medical data can be added at different times, the patient's data may be spread out across several blocks in the blockchain.

In one embodiment, the healthcare provider can identify the patient's data on a copy of the blockchain stored by computing systems operated by the healthcare provider. That is, the healthcare provider can already store a copy of the patient's data in its copy of the blockchain, which is encrypted. Thus, the healthcare provider can identify the patient's medical data from its own local copy of the blockchain.

At block 520, the healthcare provider decrypts the patient data using the key received at block 510. In this manner, a new healthcare provider can receive permission from a patient to retrieve and decrypt the patient's medical records from the blockchain. The method 500 can be used to, in a sense, transfer medical records from one healthcare provider to another. However, instead of this transfer being primarily driven by the healthcare providers, in the method 500 the patient is the one who enables the new healthcare provider to obtain her medical records from the blockchain.

Further, the healthcare providers can use the same data format such as the FHIR standard to put data in the blocks of the blockchain. As a result, the decrypted data can easily be integrated with (or added to) the patient data application used by the new healthcare provider.

In one embodiment, the method 500 can be used as part of a prophylaxis treatment or to make a medical diagnosis. As an example of making a medical diagnosis, the patient may have recently undergone a test for a particular ailment or a disease to try to diagnosis a set of symptoms experienced by the patient. In that case, the results of the test can be stored on the blockchain and then can be shared (or transferred) to a different healthcare provider to get a second opinion or to get a diagnosis.

As an example of prophylaxis treatment, the method 500 can be used to accurately transfer the patient medical data between different healthcare providers. Thus, because a human does not have to re-enter the patient's medical data when transferring the data, errors are minimized. Because the new healthcare provider has an accurate copy of the patient's medical data, this can prevent errors or mistakes when treating the patient at the new healthcare provider.

FIG. 6 is a flowchart of a method 600 for adding different types of encrypted patient data to a blockchain, according to one embodiment. As discussed in the method 500, a patient can use a key in her EHR wallet to enable a new healthcare provider to access and decrypt her medical information in the blockchain. However, the patient may want to permit the new healthcare provider to access only a certain type of her encrypted data on the blockchain. However, if all her data is encrypted using, for example, the same public key, then the same private key can be used to decrypt all her data. This means sending the private key to a new healthcare provider enables that provider to decrypt all of the patient's data.

Instead, the method 600 describes techniques for giving more granular control to the patients for their data. In other words, the patient can control how much of her medical data on the blockchain a new healthcare provider can access and decrypt. In one embodiment, different types of patient data can be encrypted using different keys which means the data is also decrypted using different keys. By controlling which keys are sent to a new healthcare provider, the patient controls what types of data the provider can access.

At block 605, a healthcare provider adds a first type of encrypted patient data for a patient to the blockchain using a first key. The first type of data can be data added during a particular time frame (e.g., the current calendar year or the current month). Or the first type of data can be treatment data (e.g., surgery, rehabilitation programs, or medication). Alternatively, the first type of data can be diagnosis data such as test results or biometric data. The embodiments herein are not limited to any particular way for dividing up a patient's medical data into different types.

At block 610, the healthcare provider adds a second type of encrypted patient data for the same patient to the blockchain using a second key. For example, the first type of data may be patient data added to the blockchain in the year 2020 while the second type of data is patient data added to the blockchain in the year 2021. Or the first type of data includes all patient data added in the previous month while the second type of data includes all patient data added in the current month. In yet another example, the first type of data may be treatment data (regardless of when it was added to the blockchain) while the second type of data is diagnosis data.

In yet another example, the first type of data may be sensitive data such as the patient's social security number, payment records, mental health records while the second type of data is less sensitive data such as a list of facilities the patient was previously admitted, dietary restrictions, biometric data, etc. In other embodiments, the second type of data may be data that the patient expects will likely be shared with a different healthcare provider, such as the results of a specific test that the healthcare provider knows will likely be sent to another healthcare provider to receive a second opinion. Encrypting this information using a different key makes it easier to transfer only the results of that specific test to the other healthcare provider without given it access to the patient's remaining healthcare records on the blockchain.

In any case, using different keys to encrypt the first and second types of the patient's data on the blockchain makes it possible for the patient to transfer only the first type of data or only the second type of data to the healthcare provider. That is, rather than simply relying on the “good will” of the healthcare provider to ensure they access only the data the patient wants them to, the patient can ensure the healthcare provider can decrypt only the type of data she wants them to.

At block 615, the healthcare provider provides respective keys to the EHR wallet for the patient to decrypt the first and second types of data. These keys may be the same as the first and second keys or may be different from the first and second keys. For example, if a public-private key pairs are used, the first and second keys may be the two public keys in the key pairs while the healthcare provider provides the two private keys to the patient to be stored in the EHR wallet.

While the method 600 describes dividing the patient's medical data into two types, the method 600 can be expanded to divide the patient's data into any number of types of data which each are encrypted using different keys. For example, a first type of data could be all patient data gathered in February that pertains to treatment data, a second type of data could be all patient data gathered in February that pertains to diagnosis data, a third type of data could be all patient data gathered in March that pertains to treatment data, a fourth type of data could be all patient data gathered in March that pertains to diagnosis data, and so forth. In this manner, the patient data can be divided using any combination of different factors, such as time, the type of medical information, the sensitivity of the medical data, and the like.

At block 620, the healthcare provider distributes the encrypted patient data to the other healthcare providers in the network. That way, these healthcare providers can add the first and second types of patient data to their copies of the distributed blockchain. As discussed above, this helps to ensure the blockchain is immutable from nefarious actors who may wish to alter or delete some of the patient data.

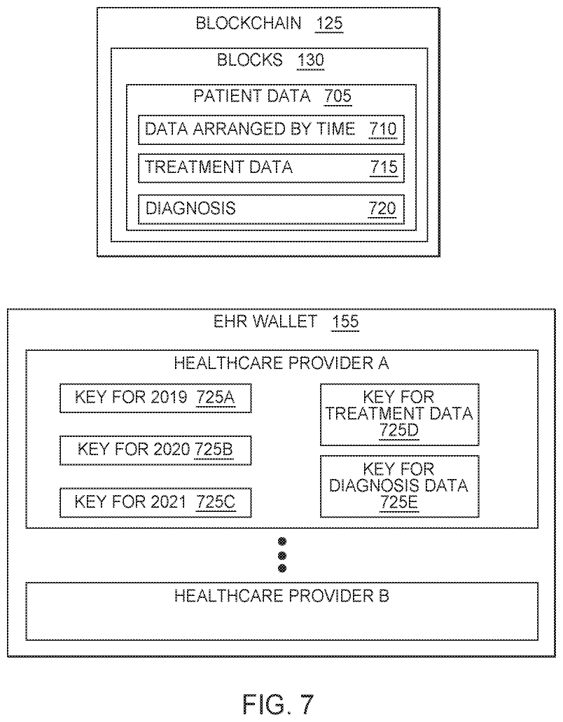

FIG. 7 illustrates a blockchain 125 and an EHR wallet 155 with keys for accessing patient data on the blockchain, according to one embodiment. In FIG. 7, the blockchain 125 includes the blocks 130 that store different types of patient data 705. Specifically, the different types of patient data are encrypted using different keys or different encryption techniques so that the patient can control whether a new healthcare provider can access some, or all, of the patient data 705.

In this example, the patient data 705 includes data arranged by time 710, treatment data, and diagnosis data 720. For example, the data arranged by time 710 could include the patient data added to the blockchain 125 at different times (e.g., all the patient data added in a year, month, or week). The treatment data 715 can include the patient data related to treating a particular disease or ailment such as medications, surgeries, rehabilitation programs, participation in addiction recovery programs, and the like. The diagnosis data 720 can include biometric data used to monitor the patient's condition (e.g., vitals, electrocardiogram, dietary restrictions, test results, etc.). The diagnosis data 520 can also list the diseases or ailments afflicting the patient.

In one embodiment, the different types of patient data 705 shown in FIG. 7 (i.e., the data arranged by time 710, treatment data 715, and diagnosis data 720) can be decrypted using different keys or encryption techniques. As such, the same key or the same encryption technique cannot be used to decrypt all of the different types of patient data 705. Instead, each of the different types of patient data may need to be decrypted using a separate key.

The EHR wallet 155 stores separate keys for decrypting the different types of patient data 705 stored in the blockchain 125. In this example, the keys stored in the EHR wallet 155 are organized based on a particular healthcare provider. That is, the EHR wallet 155 can store keys from multiple healthcare providers, assuming that multiple healthcare providers have put encrypted data for the patient on the blockchain 125.

In this case, Healthcare Provider A has encrypted patient data on the blockchain 125 for the years 2019-2021. Moreover, the Healthcare Provider A has provided separate keys for accessing the patient's treatment data and the patient's diagnosis data. In one embodiment, this data may overlap. For example, the patient data for the year 2019 may include both the treatment data and the diagnosis data generated for that patient during that year. Moreover, the key for treatment data 725D may correspond to all the treatment data generated by the patient over all three years—i.e., 2019-2021. Thus, sending the key 725A for 2019 to a new healthcare provider can enable the provider to decrypt and access all the patient data for that year (whether the data was treatment or diagnosis data), while sending the key 725D for treatment data can enable the new healthcare provider to decrypt and access all the treatment data for the patient generated by Healthcare Provider A, regardless when that treatment data was generated.

However, in another example, the data that can be decrypted by the keys 725 in the EHR wallet 155 may be non-overlapping. For example, the key 725B for 2020 may enable a new healthcare provider to decrypt any patient data that was generated in 2020 that does not include treatment and diagnosis data. For example, this information could include when the patient was admitted into a facility associated with Healthcare Provider A, how long the patient stayed at the facility, the number of times the patient visited with a healthcare professional associated with the Healthcare Provider A, or the patient's contact information.

In any case, the keys 725 enable the patient to control how much of the patient data put on the blockchain 125 by Healthcare Provider A that a new healthcare provider is able to decrypt and access. For example, if the patient wants the new healthcare provider to access all her treatment data generated by Healthcare Provider A, she can provide the key 725D for treatment data. The new healthcare provider can then use the key 725D to decrypt the treatment data for the patient put on the blockchain 125 by Healthcare Provider A. However, if Healthcare Provider B also put treatment data for the patient on the blockchain 125, the key 725D would not permit the new healthcare provider to decrypt this data. Thus, in addition to providing granular control of the different types of data put on the blockchain 125 by the same healthcare provider, the keys can also separate the data put on the blockchain by different providers as described above.

However, in another embodiment, the healthcare providers can be coordinated such that the same key can be used to decrypt a particular type of patient regardless whether that data was put on the blockchain by Healthcare Provider A or Provider B. For example, the healthcare providers can use the same public key when putting treatment data for the patient on the blockchain. The EHR wallet 155 can then store a single private key that can be used to decrypt the treatment data for the patient that was put on the blockchain by both Healthcare Providers A and B. Thus, while the embodiment shown in FIG. 7 may use different keys to permit a new healthcare provider to access a particular type of patient data put on the blockchain 125 by two different healthcare provider, in other examples, only one key may be needed to decrypt the data.

As discussed in previous embodiments, the keys 725 may be provided to the EHR wallet 155 by the Healthcare Provider A, or the keys 725 may be generated by a user device. For example, the Healthcare Provider A may generate different public-private key pairs for each of the different types of patient data 705. The Healthcare Provider A can then provide the private keys to the patient to store in the EHR wallet 155 as the keys 725. In another example, the user device for the patient can generate the key pairs and transmit the public keys to Healthcare Provider A with instructions regarding which public key to use for which types of data. This gives the patient a chance to instruct the Healthcare Provider A how to divide the patient data 705. For example, the user may want to divide the patient data in different months, rather than associating patient data from an entire year with one key pair.

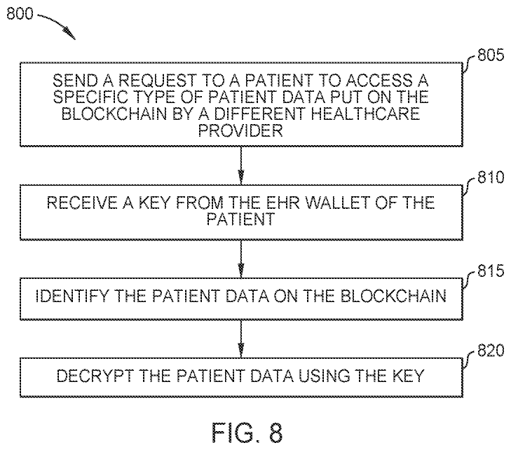

FIG. 8 is a flowchart of a method 800 for enabling a healthcare provider to access encrypted patient data on the blockchain, according to one embodiment. At block 805, a healthcare provider sends a request to a patient to access a specific type of patient data put on the blockchain by a different healthcare provider. Because the patient data was put on the blockchain by a different healthcare provider, the requesting healthcare provider would not have access to the encrypted data. For example, the patient may be seeking a second opinion from a doctor at a different healthcare provider, or the patient may be in the process of being transferred from a hospital to a long term care facility. In response, the new healthcare provider can submit a request at block 805 to the patient for access to the patient's encrypted patient data.

This request can indicate which type of data the new healthcare provider needs. For example, the new healthcare provider can indicate it only needs results for a particular test, or only diagnosis data from the last year. Thus, in one embodiment, the healthcare provider can tell the patient what type or types of patient data it needs from the blockchain. However, in other embodiments, the healthcare provider may not indicate what type of data, but rather ask for access all patient data. Nonetheless, the patient can decide to limit the types of data the new healthcare provider can access.

At block 810, the healthcare provider receives a key from the EHR wallet of the patient. The patient can evaluate the request and decide whether she wants to permit the provider to decrypt and access the specific type of data indicated in the request. For example, the healthcare provider may have requested access to the patient's medical records from the previous year, or for a specific test. The patient can then provide a key that permits the healthcare provider to decrypt a sub-portion of the patient data on the blockchain which includes the type of data requested by the healthcare provider.

In one embodiment, the patient may send multiple keys to the healthcare provider to satisfy the request. For example, the new healthcare provider may want access to all of the patient's data put on the blockchain in the last year. However, this data may be divided up into different months, where decrypting data from each month requires a different key. In that case, the patient may send the keys for all the months in the previous year to the new healthcare provider in order to satisfy the request.

Further, the patient may choose to send additional keys for data that was not requested by the new healthcare provider. For example, the healthcare provider may request access to a specific test, but the patient may know she took the same test multiple times. The patient can send the keys for the data that contains the results of those test as well. That way, the patient can tell a doctor for the new healthcare provider about the additional tests, and the doctor can immediately access the results of the other tests since the patient has sent the keys.

If the request did not stipulate what type of patient data the new healthcare provider wants, the patient can decide to limit the amount of patient data the healthcare provider can access. For example, the patient may send the key(s) that enable the new healthcare provider to access only the last two years of her medical data on the blockchain. Or the patient may send the key that permits the new healthcare provider to access only her treatment data, but not diagnosis data.

At block 815, the healthcare provider identifies the patient data on the blockchain. For example, the healthcare provider can use a search feature or labels to identify the patient's medical data on the blockchain. Because the medical data can be added at different times, the patient's data may be spread out across several blocks in the blockchain.

In one embodiment, the healthcare provider can identify the patient's data on a copy of the blockchain stored by computing systems operated by the healthcare provider. That is, the healthcare provider can already store a copy of the patient's data in its copy of the blockchain, which is encrypted. Thus, the healthcare provider can identify the patient's medical data from its own local copy of the blockchain.

At block 820, the healthcare provider decrypts the patient data using the key (or keys) received at block 810. In this manner, a new healthcare provider can receive permission from a patient to retrieve and decrypt a specific type of the patient's medical records from the blockchain. The method 800 can be used to transfer medical records from one healthcare provider to another. However, instead of this transfer being primarily driven by the old and new healthcare providers, in the method 800 the patient is the one who enables the new healthcare provider to obtain her medical records from the blockchain.

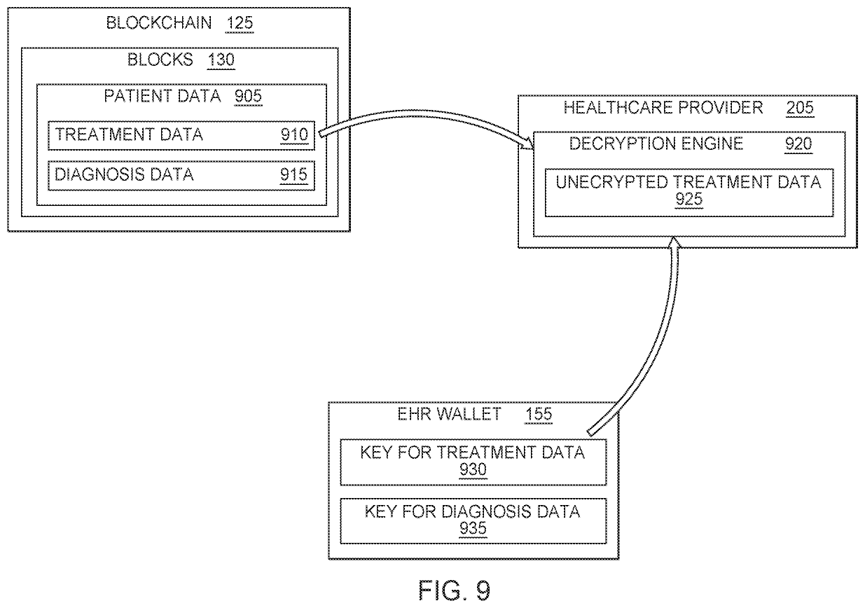

FIG. 9 illustrates decrypting different types of patient data on the blockchain, according to one embodiment. Specifically, FIG. 9 illustrates a patient enabling a healthcare provider 205 to access only a portion of the patient's data on the blockchain 125. In this example, the blocks 130 on the blockchain 125 store two different types of patient data 905: treatment data 910 and diagnosis data 915. As discussed above, the treatment data 910 can include the patient data related to treating a particular disease or ailment such as medications, surgeries, rehabilitation programs, participation in addiction recovery programs, and the like. The diagnosis data 915 can include biometric data used to monitor the patient's condition (e.g., vitals, electrocardiogram, dietary restrictions, test results, etc.).

When putting patient data 905 on the blockchain 125, a healthcare provider can determine whether the data is either treatment data 910 or diagnosis data 915 and use different encryption methods (e.g., use different public keys) when encrypting the data. The EHR wallet 155 includes a key 930 for decrypting the treatment data and a separate key 935 for decrypting the diagnosis data. As discussed above, encrypting the patient data 905 so it can only be decrypted using separate keys 930, 935 provides the patient with control when transferring the patient data to a new healthcare provider (e.g., the healthcare provider 205).

In FIG. 9, the patient has approved providing the healthcare provider 205 with access to the patient's treatment data 910. In response, the EHR wallet 155 transmits the key 930 for the treatment data to the healthcare provider 205. The healthcare provider 205 includes a decryption engine 920 which uses the key 930 to decrypt the treatment data 910 on the blockchain 125 to produce unencrypted treatment data 925. In one embodiment, the healthcare provider 205 can identify the patient's data on a copy of the blockchain 125 stored by computing systems operated by the healthcare provider 205. That is, the healthcare provider 205 can already store a copy of the patient's data in its copy of the blockchain 125, which is encrypted. Thus, the healthcare provider 205 can identify the patient's medical data from its own local copy of the blockchain 125 and decrypt the treatment data 910 for the patient using the provided key 930 to result in the unencrypted treatment data 925.

Notably, the patient has not sent the key 935 for the diagnosis data to the healthcare provider 205. Thus, while the healthcare provider 205 may have a copy of the encrypted diagnosis data 915 in its copy of the blockchain 125, the provider 205 is unable to decrypt and access the diagnosis data 915. In this manner, the patient can control what types of its patient data 905 is transferred to the healthcare provider 205.

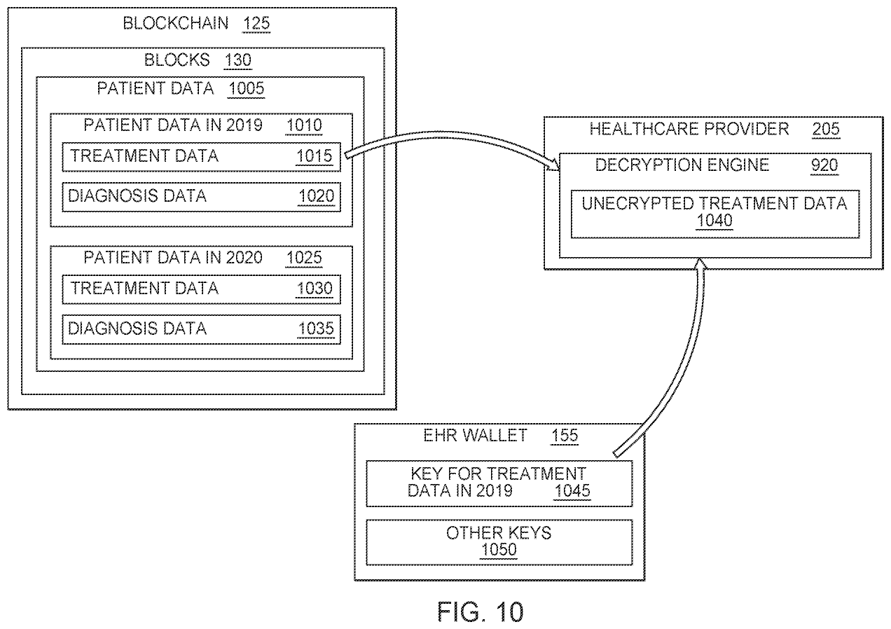

FIG. 10 illustrates decrypting different types of patient data on the blockchain, according to one embodiment. Like FIG. 9, FIG. 10 illustrates a patient enabling a healthcare provider 205 to access only a portion of the patient's data on the blockchain 125. In this example, the blocks 130 on the blockchain 125 store two different types of patient data 1005 in two different time periods. That is, the blockchain 125 stores treatment data 1015 and diagnosis data 1020 collected during 2019 and treatment data 1030 and diagnosis data 1035 collected during 2020. The healthcare provider can use different encryption methods, such as different public keys, to encrypt the treatment data 1015 collected in 2019, the diagnosis data 1020 collecting in 2019, the treatment data 1030 collected in 2020 and the diagnosis data 1035 collecting in 2020.

When putting the patient data 1005 on the blockchain 125, a healthcare provider can determine whether the data is either treatment data 1015, 1030 or diagnosis data 1020, 1035 and use different encryption methods (e.g., use different public keys) when encrypting the data. The EHR wallet 155 includes a key 1045 for decrypting the treatment data collected during 2019 and other keys 1050 for decrypting the diagnosis data 1020 collected during 2019, the treatment data 1030 collected during 2020, and the diagnosis data 1035 collected during 2020. As discussed above, encrypting the patient data 1005 so it can only be decrypted using separate keys provides the patient with control when transferring the patient data to a new healthcare provider (e.g., the healthcare provider 205).

In FIG. 10, the patient has approved providing the healthcare provider 205 with access to the patient's treatment data 1015 collected in 2019. In response, the EHR wallet 155 transmits the key 1045 for the treatment data to the healthcare provider 205. The healthcare provider 205 includes a decryption engine 920 which uses the key 1045 to decrypt the treatment data 1015 on the blockchain 125 to produce unencrypted treatment data 1040. In one embodiment, the healthcare provider 205 can identify the patient's data on a copy of the blockchain 125 stored by computing systems operated by the healthcare provider 205. That is, the healthcare provider 205 can already store a copy of the patient's data in its copy of the blockchain 125, which is encrypted. Thus, the healthcare provider 205 can identify the patient's medical data from its own local copy of the blockchain 125 and decrypt the treatment data 1015 for the patient using the provided key 1045 to result in the unencrypted treatment data 1040.

Notably, the patient has not sent the other keys 1050 for decrypting the diagnosis data 1020 collected in 2019, the treatment data 1030 collect in 2020, and the diagnosis data 1035 collected in 2020 to the healthcare provider 205. Thus, while the healthcare provider 205 may have a copy of the encrypted diagnosis data 1020, the treatment data 1030, and the diagnosis data 1035 in its copy of the blockchain 125, the provider 205 is unable to decrypt and access this data. In this manner, the patient can control what types of its patient data 1010 is transferred to the healthcare provider 205.

FIG. 11 is a flowchart of a method 1100 for enabling an organization to access encrypted patient data on the blockchain, according to one embodiment. At block 1105, the organization transmits an offer to a patient to access the patient's encrypted data on the blockchain. Method 1100 assumes the organization did not put the patient's data on the blockchain, and thus, does not have access to the data. For example, the organization may be verified academic, research or private organization that wants to use the patient's data to perform research, medical analysis, and the like. In another example, the organization may be the healthcare provider that added the patient's medical data onto the blockchain, but might want to use that data for a different purpose besides treating the patient, such as performing research. As such, the healthcare provider may ask for the patient's permission for using the data for a different reason.

In one embodiment, the offer includes a payment to the patient for access to her data. The payment could be a monetary payment (e.g., a dollar amount), a discount code or coupon, a gift, and the like. Thus, the patient can decide whether to accept the payment, and grant access to her medical data, or not. Further, the offer can indicate that any identifying data in the medical data will be removed

In one embodiment, the offer can be presented to the patient using the EHR wallet. For example, the EHR wallet can have a function to display offers from organizations, along with payments corresponding to the offers. The patient can then select, using a graphical user interface (GUI) which offers to accept and which to deny. For example, the patient may not trust certain organizations, or may feel the payment is too low. The patient may be able to send a counter offer to the organization to increase the payment using the EHR wallet.

At block 1110, if the patient rejects the offer, the method 1100 ends. If the patient accepts the offer, the method proceeds to block 1115 where the organization provides the payment to the patient. The payment could be provided using an automatic delivery such as displaying a coupon code or transferring funds to a pre-designated account or using other delivery means such as mailing the patient a gift card.

The method 1100 can then perform blocks 810, 815, and 820 of the method 800 where the organization receives a key (or keys) from the EHR wallet of the patient to decrypt the patient data. As discussed above, the key (or keys) can permit the organization to decrypt only a portion of the patient data on the blockchain.

At block 1120, the organization removes identifying information from the decrypted patient data. For example, as part of the offer, the organization may promise the patient it will first parse the data to remove any identifying information such as name, social security number, telephone number, payment information, and the like. After decrypting the patient data, the organization can parse the data to remove and discard this sensitive information. That is, the organization only saves non-identifying or non-sensitive medical data for the patient which it can then use to perform research, analyze, and the like.

### Example Computing Hardware

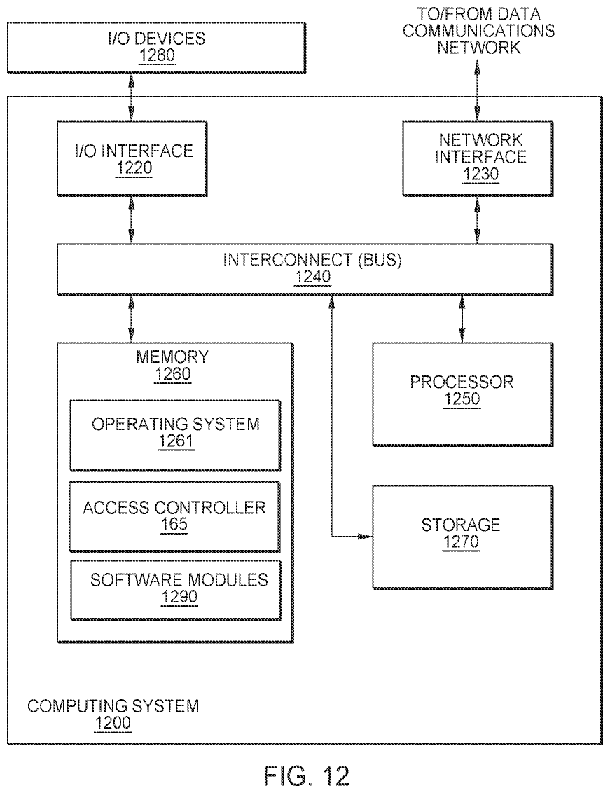

FIG. 12 illustrates a computing system 1200, which may be used to implement the computing systems 210 in FIG. 2 or the user device 150 in FIG. 1 (e.g., a computer, a laptop, a tablet, a smartphone, web server, data center, cloud computing environment, etc.), or any other computing device described in the present disclosure. As shown, the computing system 1200 includes, without limitation, a computer processor 1250 (e.g., a central processing unit), a network interface 1230, and memory 1260. The computing system 1200 may also include an input/output (I/O) device interface 1220 connecting I/O devices 1280 (e.g., keyboard, display and mouse devices) to the computing system 1200.

The processor 1250 retrieves and executes programming instructions stored in the memory 1260 (e.g., a non-transitory computer readable medium). Similarly, the processor 1250 stores and retrieves application data residing in the memory 1260. An interconnect 1240 facilitates transmission, such as of programming instructions and application data, between the processor 1250, I/O device interface 1220, storage 1270, network interface 1230, and memory 1260. The processor 1250 is included to be representative of a single processor, multiple processors, a single processor having multiple processing cores, and the like. And the memory 1260 and the storage 1270 are generally included to be representative of volatile and non-volatile memory elements. For example, the memory 1260 and the storage 1270 can include random access memory and a disk drive storage device. Although shown as a single unit, the memory 1260 or the storage 1270 may be a combination of fixed and/or removable storage devices, such as magnetic disk drives, flash drives, removable memory cards or optical storage, network attached storage (NAS), or a storage area-network (SAN). The storage 1270 may include both local storage devices and remote storage devices accessible via the network interface 1230. If the computing system 1200 is used as a user device 150 in FIG. 1, the access controller 165 is maintained in the memory 1260 to maintain and transmit the keys to new healthcare providers to transfer the patient's medical data on the blockchain. Additionally, one or more software modules 1290 may be maintained in the memory to perform the functions of an EHR wallet or decryption engine as discussed above.

Further, the computing system 1200 is included to be representative of a physical computing system as well as virtual machine instances hosted on a set of underlying physical computing systems. Further still, although shown as a single computing device, one of ordinary skill in the art will recognize that the components of the computing system 1200 shown in FIG. 12 may be distributed across multiple computing systems connected by a data communications network.

As shown, the memory 1260 includes an operating system 1261. The operating system 1261 may facilitate receiving input from and providing output to various components. For example, the network interface 1230 can be used to output encrypted data to the blockchain or transmit and receive keys discussed above. In another embodiment, the computing system 1200 can be used to store a copy of the blockchain.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

Clause 1: A method, comprising transmitting, from a first healthcare provider, a request to access encrypted patient data put on a blockchain by a second healthcare provider; receiving a cryptography element from an electronic health record (EHR) wallet controlled by a recipient of the request; identifying the encrypted patient data on the blockchain; and decrypting the encrypted patient data using the cryptography element.

Clause 2: In addition to the clause 1, wherein the blockchain is distributed between the first and second healthcare providers.

Clause 3: In addition to the clauses 1 or 2, wherein the blockchain comprises other encrypted patient data for patients of both the first healthcare provider and the second healthcare provider.

Clause 4: In addition to the clause 1, 2, or 3, wherein the blockchain comprises different encrypted patient data for a same patient corresponding to the encrypted patient data, wherein the different encrypted patient data that was put on the blockchain by the second healthcare provider, wherein the EHR wallet contains a second cryptography element for decrypting the different encrypted patient data.

Clause 5: In addition to the clause 4, wherein the encrypted patient data and the different encrypted patient data comprises different types of medical data corresponding to the patient.

Clause 6: In addition to the clause 5, wherein the different types of medical data comprise one of: medical data generated at different times by the second healthcare provider, treatment data for the patient, or diagnosis data for the patient.

Clause 7: In addition to the clauses 5, or 6 wherein the request indicates which of the different types of medical data is being requested by the first healthcare provider, wherein the encrypted patient data corresponds to the type of medical data being requested by the first healthcare provide.

Clause 8: A method, comprising: adding, by a first healthcare provider, first encrypted patient data for a first patient to a first block in a blockchain; adding, by the first healthcare provider, second encrypted patient data for a second patient to a second block in the blockchain; and distributing the blockchain, which contains the first and second blocks, to a plurality of healthcare providers.

Clause 9: In addition to the clause 8, wherein the first encrypted patient data is encrypted using a different encryption key than the second encrypted patient data.

Clause 10: In addition to the clauses 8 or 9, further comprising: providing a first private key for decrypting the first encrypted patient data to a first EHR wallet; and providing a second private key for decrypting the second encrypted patient data to a second EHR wallet.

Clause 11: In addition to the clause 10, wherein the first encrypted patient data is encrypted using a first public key that is part of a first key pair with the first private key and the second encrypted patient data is encrypted using a second public key that is part of a second key pair with the second private key.

Clause 12: In addition to the clauses 8, 9, 10, or 11, further comprising: identifying, by the first healthcare provider, a first type of medical data of the first patient, wherein the first type of medical data is encrypted to form the first encrypted patient data; and identifying, by the first healthcare provider, a second type of medical data of the first patient; encrypting the second type of medical data to form third encrypted patient data for the first patient; and adding, by the first healthcare provider, the third encrypted patient data to a third block in the blockchain.

Clause 13: In addition to the clause 12, further comprising: providing a first key for decrypting the first encrypted patient data to a first EHR wallet; and providing a second key for decrypting the third encrypted patient data to the first EHR wallet.

Clause 14: In addition to the clause 13, wherein the first type of medical data is encrypted using a first public key that is part of a first key pair with the first key and the second type of medical data is encrypted using a second public key that is part of a second key pair with the second key.

Clause 15: A non-transitory computer readable medium comprising instructions to be executed in a processor, the instructions when executed in the processor perform an operation, the operation comprising: transmitting, from a first healthcare provider, a request to access encrypted patient data put on a blockchain by a second healthcare provider; receiving a cryptography element from an EHR wallet controlled by a recipient of the request; identifying the encrypted patient data on the blockchain; and decrypting the encrypted patient data using the cryptography element.

Clause 16: In addition to the clause 15, wherein the blockchain is distributed between the first and second healthcare providers.

Clause 17: In addition to the clauses 15 or 16, wherein the blockchain comprises other encrypted patient data for patients of both the first healthcare provider and the second healthcare provider.

Clause 18: In addition to the clauses 15, 16, or 17, wherein the blockchain comprises different encrypted patient data for a same patient corresponding to the encrypted patient data, wherein the different encrypted patient data that was put on the blockchain by the second healthcare provider, wherein the EHR wallet contains a second cryptography element for decrypting the different encrypted patient data.

Clause 19: In addition to the clause 18, wherein the encrypted patient data and the different encrypted patient data comprises different types of medical data corresponding to the patient.

Clause 20: In addition to the clause 19, wherein the request indicates which of the different types of medical data is being requested by the first healthcare provider, wherein the encrypted patient data corresponds to the type of medical data being requested by the first healthcare provider.

Clause 21: A method comprising: adding, by a first healthcare provider, first encrypted patient data for a first patient to a first block in a blockchain; providing a key to a second healthcare provider or a group of healthcare providers to grant access to the first encrypted patient data; and distributing the blockchain, which contains the first block, to a plurality of healthcare providers.

Clause 22: In addition to the clause 21, wherein the second healthcare provider or a group of healthcare providers have a preexisting relationship with the first patient.

Clause 23: In addition to the clause 21, the method further comprising: storing a treatment recommendations from the group of healthcare providers on the blockchain; and determining, using the treatment recommendations on the blockchain, whether a consensus has been reached among the group of healthcare providers.

What is claimed is:

1. A method, comprising:
transmitting, from a first healthcare provider, a request to access encrypted patient data put on a blockchain by a second healthcare provider;
receiving a cryptography element from an electronic health record (EHR) wallet controlled by a recipient of the request;
identifying the encrypted patient data on the blockchain; and
decrypting the encrypted patient data using the cryptography element.

2. The method of claim 1, wherein the blockchain is distributed between the first and second healthcare providers.

3. The method of claim 1, wherein the blockchain comprises other encrypted patient data for patients of both the first healthcare provider and the second healthcare provider.

4. The method of claim 1, wherein the blockchain comprises different encrypted patient data for the same patient corresponding to the encrypted patient data, wherein the different encrypted patient data was put on the blockchain by the second healthcare provider,
wherein the EHR wallet contains a second cryptography element for decrypting the different encrypted patient data.

5. The method of claim 4, wherein the encrypted patient data and the different encrypted patient data comprises different types of medical data corresponding to the patient.

6. The method of claim 5, wherein the different types of medical data comprise one of: medical data generated at different times by the second healthcare provider, treatment data for the patient, or diagnosis data for the patient.

7. The method of claim 5, wherein the request indicates which of the different types of medical data is being requested by the first healthcare provider, wherein the encrypted patient data corresponds to the type of medical data being requested by the first healthcare provider.

8. A method, comprising:
adding, by a first healthcare provider, first encrypted patient data for a first patient to a first block in a blockchain;
adding, by the first healthcare provider, second encrypted patient data for a second patient to a second block in the blockchain; and
distributing the blockchain, which contains the first and second blocks, to a plurality of healthcare providers.

9. The method of claim 8, wherein the first encrypted patient data is encrypted using a different encryption key than the second encrypted patient data.

10. The method of claim 8, further comprising:
providing a first private key for decrypting the first encrypted patient data to a first EHR wallet; and
providing a second private key for decrypting the second encrypted patient data to a second EHR wallet.

11. The method of claim 10, wherein the first encrypted patient data is encrypted using a first public key that is part of a first key pair with the first private key, and the second encrypted patient data is encrypted using a second public key that is part of a second key pair with the second private key.

12. The method of claim 8, further comprising:
identifying, by the first healthcare provider, a first type of medical data of the first patient, wherein the first type of medical data is encrypted to form the first encrypted patient data; and
identifying, by the first healthcare provider, a second type of medical data of the first patient;
encrypting the second type of medical data to form third encrypted patient data for the first patient; and
adding, by the first healthcare provider, the third encrypted patient data to a third block in the blockchain.

13. The method of claim 12, further comprising:
providing a first key for decrypting the first encrypted patient data to a first EHR wallet; and
providing a second key for decrypting the third encrypted patient data to the first EHR wallet.

14. The method of claim 13, wherein the first type of medical data is encrypted using a first public key that is part of a first key pair with the first key and the second type of medical data is encrypted using a second public key that is part of a second key pair with the second key.

15. A non-transitory computer readable medium comprising instructions to be executed in a processor, the instructions when executed in the processor perform an operation, the operation comprising:
transmitting, from a first healthcare provider, a request to access encrypted patient data put on a blockchain by a second healthcare provider;
receiving a cryptography element from an EHR wallet controlled by a recipient of the request;
identifying the encrypted patient data on the blockchain; and
decrypting the encrypted patient data using the cryptography element.

16. The non-transitory computer readable medium of claim 15, wherein the blockchain is distributed between the first and second healthcare providers.

17. The non-transitory computer readable medium of claim 15, wherein the blockchain comprises other encrypted patient data for patients of both the first healthcare provider and the second healthcare provider.

18. The non-transitory computer readable medium of claim 15, wherein the blockchain comprises different encrypted patient data for the same patient corresponding to the encrypted patient data, wherein the different encrypted patient data was put on the blockchain by the second healthcare provider,
wherein the EHR wallet contains a second cryptography element for decrypting the different encrypted patient data.

19. The non-transitory computer readable medium of claim 18, wherein the encrypted patient data and the different encrypted patient data comprises different types of medical data corresponding to the patient.

20. The non-transitory computer readable medium of claim 19, wherein the request indicates which of the different types of medical data is being requested by the first healthcare provider, wherein the encrypted patient data corresponds to the type of medical data being requested by the first healthcare provider.

21. A method, comprising:
adding, by a first healthcare provider, first encrypted patient data for a first patient to a first block in a blockchain;
providing a key to a second healthcare provider or a group of healthcare providers to grant access to the first encrypted patient data; and
distributing the blockchain, which contains the first block, to a plurality of healthcare providers.

22. The method of claim 21, wherein the second healthcare provider or a group of healthcare providers have a preexisting relationship with the first patient.

23. The method of claim 21, further comprising:
storing a treatment recommendations from the group of healthcare providers on the blockchain; and
determining, using the treatment recommendations on the blockchain, whether a consensus has been reached among the group of healthcare providers.
